(19) United States
(12) Patent Application Publication
(10) Pub. No.: US 2025/0135094 A1
(43) Pub. Date: May 1, 2025

(54) NASAL IRRIGATOR SYSTEM WITH REAL-TIME CONGESTION MAPPING AND AI-BASED MOBILE INTEGRATION

(71) Applicant: Amor Laala, Abu Dhabi (AE)

(72) Inventor: Amor Laala, Abu Dhabi (AE)

(21) Appl. No.: 19/004,361

(22) Filed: Dec. 29, 2024

(51) Int. Cl. A61M 3/02; G16H 20/13; G16H 50/20

(52) U.S. Cl. CPC A61M 3/0202; A61M 3/0258; G16H 20/13; G16H 50/20; A61M 2202/0007; A61M 2202/04; A61M 2205/18; A61M 2205/3327; A61M 2205/3334; A61M 2205/3561; A61M 2205/3576; A61M 2205/502; A61M 2205/52; A61M 2210/0618; A61M 2230/42

(57) ABSTRACT

A nasal irrigation monitoring and congestion mapping system is disclosed herein. The system comprises an electrical nasal irrigation device and a mobile application. In an embodiment, the electrical nasal irrigation device includes an electronic pump circuit having a microcontroller unit (MCU) configured to control the operation of the pump and pressure modes and a pressure detection sensor to measure the flow rate of saline solution to identify pressure fluctuation data in the nasal passages during saline delivery, and provide real-time data on nasal congestion levels. A mobile application is communicatively connected to the nasal irrigation device to receive and interpret the pressure fluctuation data using congestion mapping algorithm to assess a degree of nasal congestion, and map the congestion severity as nasal congestion data based on the degree of nasal congestion.

### FIELD OF THE INVENTION

The present invention relates to nasal irrigation system and more particularly relates to nasal irrigation system with real-time congestion mapping and AI-based mobile integration.

### BACKGROUND OF THE INVENTION

Nasal irrigation devices are widely regarded as an essential component of respiratory health management, offering therapeutic benefits for specific conditions such as chronic sinusitis, allergic rhinitis, and post-operative care following sino-nasal surgeries. By effectively removing mucus, allergens, and other irritants, these devices help alleviate nasal congestion, reduce inflammation, and improve airflow, leading to their widespread adoption in both clinical and home care settings.

Existing nasal irrigation devices primarily consist of mechanical systems, ranging from traditional gravity-based solutions like Neti pots to more modern battery-operated pulsating irrigators. While these devices have proven beneficial for many users, they operate on a simple principle of fluid delivery, lacking the capability to adapt to individual user needs or specific nasal conditions. This standardized approach, though effective in many cases, does not address the highly variable nature of nasal congestion or the diverse anatomical differences among users, limiting its overall effectiveness.

The limitations of existing nasal irrigation devices become particularly apparent in their inability to provide objective measurement or monitoring of treatment effectiveness. Users are forced to rely solely on subjective assessments of their symptoms, with no means to track progress or optimize their treatment regimen using quantitative data. This lack of monitoring capability also hinders healthcare providers from making data-driven decisions regarding treatment modifications or gaining valuable insights into treatment adherence and long-term effectiveness.

Current devices lack a mechanism for real-time monitoring of congestion levels and providing real-time feedback, such as data-driven insights and predictive alerts about congestion. Furthermore, these devices are not adaptable to a user's specific congestion condition. Implementing such a system could significantly improve treatment outcomes and offer valuable data for users, particularly those with chronic sinusitis or allergies, enabling optimized respiratory health management.

Therefore, there is a need for a nasal irrigation system capable of real-time monitoring, providing congestion feedback, and delivering predictive alerts, all while optimizing respiratory health management.

### BRIEF SUMMARY OF THE INVENTION

It is an object of the present invention to provide a nasal irrigation device with integrated real-time congestion monitoring for enhanced respiratory health management.

Another object of the present invention is to provide a nasal irrigation system that accurately detects and maps nasal congestion using pressure fluctuation measurements, offering an intuitive user interface for improved usability.

Another object of the present invention is to enable tracking and analysis of historical nasal congestion patterns using a mobile application.

It is also an object of the present invention to provide personalized health insights and recommendations for nasal irrigation schedules through the analysis of historical congestion data using artificial intelligence.

Another object of the present invention is to incorporate an AI-based predictive module to analyze real-time congestion data trends and provide actionable insights.

According to an embodiment of the present invention, a nasal irrigation monitoring and congestion mapping system is disclosed. The system includes an electrical nasal irrigation device comprising a water reservoir configured to store a saline solution. A pump connected to the reservoir delivers the solution to the user's nasal passages. The device features an electronic pump circuit equipped with a microcontroller unit (MCU) to regulate the pump's operation and pressure modes. A pressure detection sensor, connected to the MCU via an analog-to-digital converter (ADC), measures nasal congestion by detecting pressure fluctuations during saline delivery. A mobile application communicates with the nasal irrigation device via the MCU and is programmed with a congestion mapping algorithm to analyze the sensor data, interpret pressure fluctuations, and generate a severity map of congestion. The system also features a graphical user interface (GUI) to display the congestion map as a heatmap, visually highlighting areas of significant nasal blockage.

### BRIEF DESCRIPTION OF DRAWINGS

The present invention will become clearly understood to those of ordinary skill in the art when descriptions of exemplary embodiments thereof are read with reference to the accompanying drawings.

FIG. 1 is a schematic view of a nasal irrigation device, depicting key components according to an embodiment of the present invention.

FIG. 2 is a schematic block diagram of a part of a nasal irrigation monitoring and congestion mapping system depicting a pump circuit and a mobile application.

FIG. 3 is a user interface of the mobile application of FIG. 2 depicting the congestion level data as charts.

FIG. 4 is a user interface of FIG. 3 including visual indicators such as connectivity status, session pause/resume options, and congestion history.

FIG. 5 is a graphical representation of the congestion history depicted in the user interface of FIG. 3.

### DETAILED DESCRIPTION OF THE INVENTION

The novel features which are believed to be characteristic of the invention, both as to its organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present invention. For a more complete understanding of the present invention, reference is now made to the following descriptions taken in conjunction with the accompanying drawings.

It is to be understood that the terms “comprising,” “comprises,” “includes,” “containing,” “having,” and “featuring,” as used throughout this description, are to be interpreted as open-ended terms. These terms indicate the presence of the stated elements, features, or steps but do not exclude the possibility of additional elements, features, or steps. Their use ensures that the claims cover embodiments with both the explicitly mentioned features and other features that do not detract from the overall functionality or purpose of the invention.

It is to be understood that the term “Artificial Intelligence (AI),” as used throughout this description, refers broadly to computational systems, algorithms, and methodologies that enable machines to perform tasks typically requiring human intelligence. This includes, but is not limited to, machine learning, deep learning, natural language processing, neural networks, computer vision, expert systems, rule-based reasoning, and any other techniques, methods, or algorithms designed to mimic, replicate, or enhance cognitive functions such as learning, problem-solving, decision-making, perception, and adaptation.

The use of the term “AI” should not be construed as being limited to any specific technology, architecture, or implementation approach. It encompasses a wide range of techniques, whether currently known or developed in the future, that achieve the described functions or objectives. Furthermore, the term is not limited to autonomous systems and can also include semi-autonomous or manually supervised systems that incorporate AI-based components.

The present invention pertains to a nasal irrigation monitoring and congestion mapping system, described through various embodiments. In an exemplary embodiment, the system integrates a nasal irrigation device with pressure detection capabilities and a mobile application for tracking and analyzing nasal congestion. The system provides real-time monitoring and feedback by measuring pressure fluctuations during irrigation to identify areas of nasal blockage. Furthermore, the invention leverages artificial intelligence (AI) to analyze historical congestion trends and deliver predictive insights for improved long-term respiratory health management.

In some embodiments, the nasal irrigation device includes a water reservoir for storing saline solution, a pump to deliver the solution to the nasal passages, and an electronic pump circuit with a microcontroller unit (MCU) that controls the pump's operation. A pressure detection sensor measures the flow rate of saline solution and pressure fluctuations within the nasal passages during saline delivery for enabling congestion detection. This pressure data is transmitted to a mobile application via the MCU, where it is analyzed and displayed. In an exemplary aspect, the mobile application includes a congestion mapping algorithm that interprets the pressure data to generate a congestion map. A graphical user interface (GUI) presents this congestion map as a heatmap for users to visualize areas of nasal blockage. The application further comprises a database to store historical congestion data, and an AI-powered predictive analysis module that uses stored data to identify trends, forecast potential health conditions, and suggest personalized feedback for the user.

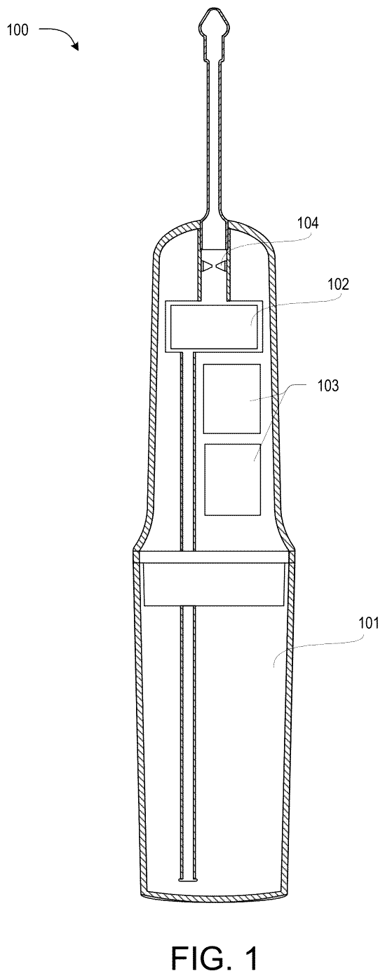

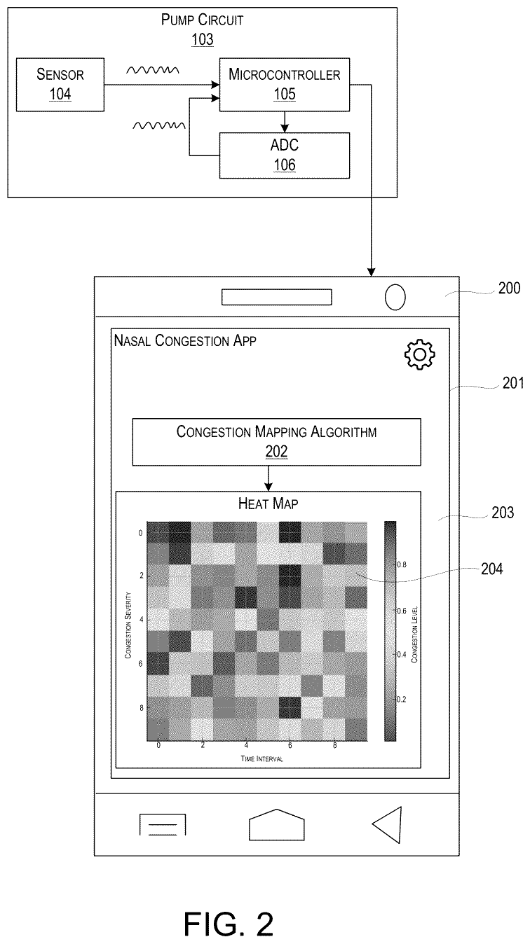

According to an exemplary embodiment of the present invention, a nasal irrigation monitoring and congestion mapping system, hereinafter referred to as the “system,” is disclosed. The system includes a nasal irrigation device 100 which is shown in FIG. 1 and a mobile application 201 which is shown in FIG. 2. The nasal irrigation device 100 comprises a water reservoir 101, a pump 102, an electronic pump circuit 103 and a pressure detection sensor 104. The mobile application 201 includes congestion mapping algorithm 202, an artificial intelligence (AI) predictive analysis module and a database. In one embodiment, the pressure detection sensor 104 is configured as a part of the electronic pump circuit 103. In another embodiment, the pressure detection sensor 104 is configured as an external element, however, connected to the electronic pump circuit 103.

In an exemplary aspect, the water reservoir 101 is configured as a sealed water reservoir 101 and is configured to store a saline solution or appropriate liquid to be irrigated to the nostrils of a user. The water reservoir 101 is refillable and maintains the appropriate saline concentration for optimal nasal irrigation. The reservoir 101 is made from a medical-grade, biocompatible material to prevent contamination and ensure safe use. It is designed with an airtight seal to maintain the purity of the saline solution, preventing any contaminants from entering the stored liquid. In some aspects, the reservoir 101 includes a removable, leak-proof cap that allows for easy refilling and secure closure. The reservoir 101 further includes measurement markings that guide the user in filling it with the appropriate volume of saline solution for consistent irrigation. The reservoir 101 is also made of a material that is durable and resistance to repeated sterilization to ensure long-term usability without degradation. In an exemplary aspect, the reservoir 101 is made of a transparent glass or medical-grade plastic material. The reservoir of present invention is of varying size in order to support varying user needs. Additionally, a flow control mechanism (not shown) may be configured in the present invention in order to regulate the release of saline solution for consistent flow and prevent backflow during irrigation, which enhances both safety and the effectiveness of nasal cleaning.

The pump 102 is connected to the water reservoir to deliver the saline solution to the nasal passages under controlled pressure. The pump's delivery mode can be adjusted by means of the flow control mechanism based on the user's comfort level and the severity of nasal blockage. In an exemplary aspect, the electronic pump circuit 103 includes a microcontroller unit (MCU) 105 that controls pump operation, pressure levels, and different irrigation modes in connection with the flow control mechanism. The MCU 105 enables precise control over the saline delivery to the nasal passages for effective congestion management. In an exemplary embodiment, the pressure detection sensor 104 is communicatively connected to the MCU 105 through an analog-to-digital converter (ADC) 106.

In an exemplary embodiment, the pressure detection sensor 104 is configured to measure the flow rate of saline solution to identify pressure fluctuations in the nasal passages during saline delivery, and provide real-time data on nasal congestion levels. The sensor 104 detects resistance in the nasal passages, while saline solution passes through the nasal passages, to interpret the pressure fluctuations as indicators of congestion. In other words, lower the flow rate of saline solution, higher the congestion level, and vice versa.

In some embodiments, the pressure fluctuation data (raw data) is amplified using an amplifier (not shown) and filtered using low-pass or high-pass filters (not shown), to remove noise and unwanted artifacts, wherein the amplifier and filters are part of the pump circuit 103 and connected to the MCU 105. Upon filtering, the data is segmented into inhalation and exhalation phases, allowing for a more detailed analysis of the breathing cycle. The frequency and amplitude of the pressure fluctuations are then analyzed to detect changes caused by nasal blockages, where reduced airflow indicates higher resistance and, consequently, greater congestion. In some embodiments, the filtered pressure fluctuation data is converted to digital data using the ADC 106 for simplified analysis by the mobile application 201. Also, as the amplifier amplifies the data signals, signal quality that the mobile application 201 receives is enhanced, thereby allowing for more accurate digital interpretation by the MCU 105 and enhanced congestion mapping precision. The digitally converted pressure data is transmitted to mobile application 201 via the MCU 105.

In an exemplary embodiment, the mobile application 201 is communicatively connected to the nasal irrigation device 100 to receive pressure data via the MCU 105. In one aspect, the mobile application in the context of the congestion mapping system is a software program installed on a mobile device 200, including but not limited to a smartphone, computer, laptop or tablet, which functions as a central hub for data collection, analysis, and visualization. The application 201 communicates wirelessly with the nasal irrigation device 100 using communication technologies such as Bluetooth or Wi-Fi, allowing it to receive real-time digital pressure fluctuation data. Once the data is transmitted to the application 201, it processes the information using an integrated congestion mapping algorithm 202 that evaluates airflow patterns and assesses nasal congestion levels.

As aforementioned, the application 201 is programmed with congestion mapping algorithm 202 for processing pressure fluctuation data measured by the pressure detection sensor 104. The congestion mapping algorithm 202 interprets the pressure fluctuation data to assess the degree of nasal congestion. Based on the interpreted data, the algorithm 202 generates a map of nasal congestion, and identifies higher or lower congestion levels. In an exemplary embodiment, the algorithm 202 extracts features, i.e. key parameter, from the digital pressure data, for assessing nasal congestion. In one embodiment, the key parameter includes pressure amplitude, representing the intensity of airflow; lower amplitudes correlate with greater nasal obstruction. Additionally, the pressure gradient, which measures the rate of pressure change over time, helps determine the level of resistance to airflow. The algorithm 202 also considers frequency of breathing cycles, using the timing between pressure peaks and troughs to identify irregular patterns that may indicate impaired nasal airflow.

In specific embodiment, the extracted features are used to evaluate congestion levels by analyzing the relationship between time and pressure fluctuations. For instance, prolonged cycles with reduced pressure gradients suggest higher airflow resistance and severe congestion. To quantify this, the algorithm 202 computes congestion severity scores by comparing features to predefined thresholds derived from clinical data or user-specific baselines. This temporal analysis enables the system to classify airflow patterns into clear, mildly congested, or highly congested states, providing a precise assessment without the need for spatial mapping of nasal zones.

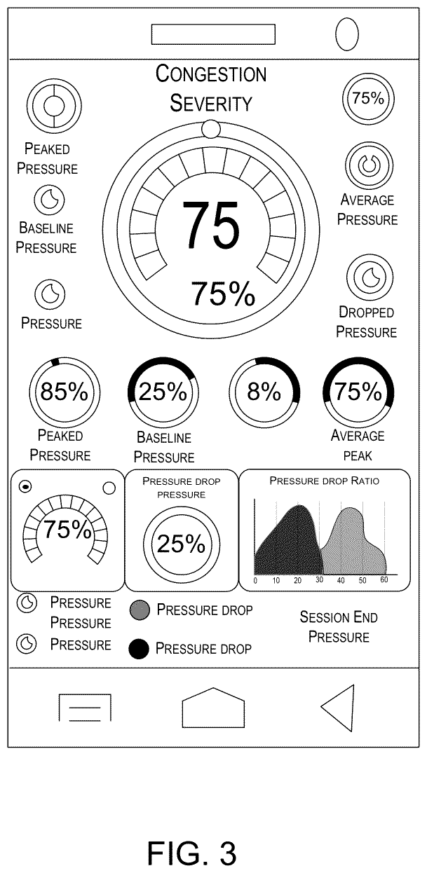

In some embodiments, the mobile application 201 employs the congestion severity scores generated by the algorithm 202 to create a dynamic heatmap 204 that visually represents, in a graphical user interface (GUI) 203, nasal airflow patterns over time. An example congestion severity graph is shown in FIG. 3. These scores, derived from time-pressure data, quantify the degree of congestion during each breathing cycle. The severity scores are normalized to a predefined range, such as 0 (no congestion) to 100 (maximum congestion), ensuring consistency and accuracy in visual representation.

The heatmap 204 uses a temporal framework to structure the data. The x-axis represents the timeline of the pressure readings, segmented into time intervals corresponding to individual breathing cycles or user-defined durations. The y-axis, on the other hand, denotes congestion severity, scaled according to the normalized scores. Each data point is plotted on the heatmap grid, correlating its timestamp with the assessed congestion level. This temporal mapping enables a clear visualization of congestion trends over time.

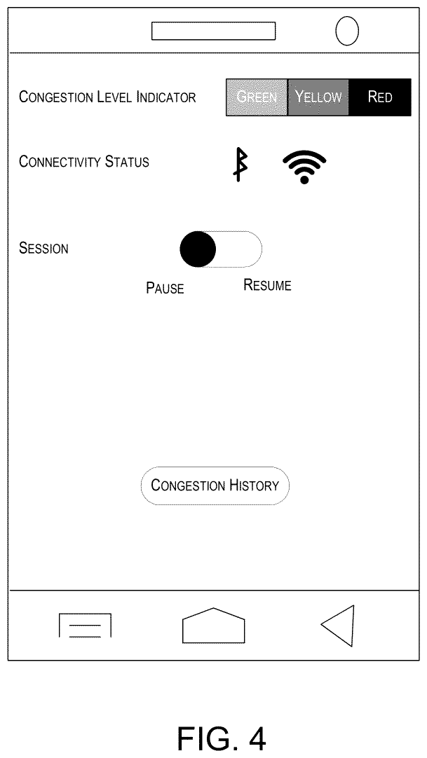

Referring FIG. 4, color gradients are used to encode the congestion severity for intuitive understanding. For instance, areas with low congestion are marked in green, indicating clear airflow, while yellow signifies moderate congestion. Regions of high congestion are represented in red, highlighting significant nasal blockage. The smooth color transitions within the gradient provide a comprehensive overview of varying congestion levels, aiding in the detection of patterns or anomalies.

The heatmap 204 is dynamically rendered in real-time, updating continuously as new pressure data is analyzed. This allows users to observe changes in nasal airflow patterns as they occur. The GUI 203 enhances user interaction by providing features such as zooming into specific time intervals for detailed examination, displaying numerical congestion scores for selected data points, and toggling between live updates and historical data for comparative analysis.

In some embodiments, additional visual enhancements, such as event markers for sneezing or nasal spray use, can be overlaid on the heatmap 204. These markers provide context for variations in congestion, enabling users to correlate actions or environmental factors with changes in nasal airflow.

In specific aspects, the mobile application 201 includes a database that stores historical congestion data. Using the historical congestion data, the user is allowed to track congestion trends over time for chronic nasal conditions and receive insights on past and current congestion levels for further medical consultations if needed. In an exemplary aspect, the AI predictive analysis module is connected to the database and is configured with machine learning algorithm 202 to analyze congestion data trends over time. The machine learning algorithm 202 helps identify patterns suggestive of specific conditions, such as sinus infections or seasonal allergies. The AI predictive analysis module is also configured to provide personalized insights, such as recommended irrigation schedules and alerts for recurring congestion patterns that may require medical attention. In one specific aspect, based on historical congestion patterns stored in the database, the AI predictive analysis module generates personalized recommendations, including optimal irrigation frequencies. Additionally, users are also alerted of potential nasal health issues, such as sinus infections or allergy flare-ups, and suggest preventive measures.

The AI predictive module identifies critical congestion patterns that may indicate underlying nasal health conditions specifically by identifying patterns associated with recurring infections, allergy onset, or chronic sinus issues. By analyzing aforementioned trends, the system helps user enhance their respiratory health.

In a specific aspect, the system is connected to a stable power supply to transmit power to the electronic pump circuit 103. As consistent voltage reduces signal noise, the accuracy of pressure measurements is further improved. In some aspects, the mobile application 201 is further integrated with cloud-based storage for secure, long-term data retention and analysis.

According to an embodiment, when the user irrigates liquid via the nasal irrigation pump 102, the pump 102 delivers the saline solution through the nasal passages. The pressure detection sensor 104 measures resistance in the nasal cavities, while accurately capturing pressure fluctuations that indicate varying congestion levels. The sensor 104 sends pressure fluctuation data to the MCU 105, which transmits it to the mobile application 201. The pressure fluctuation data is measured as analog data. In an exemplary aspect, before transmitting the pressure fluctuation data, the MCU 105 processes the data using the amplifier, filters and the ADC 106. The amplifier is configured to amplify the pressure fluctuation data and filters remove noise in the pressure data before transmission to the ADC 106.

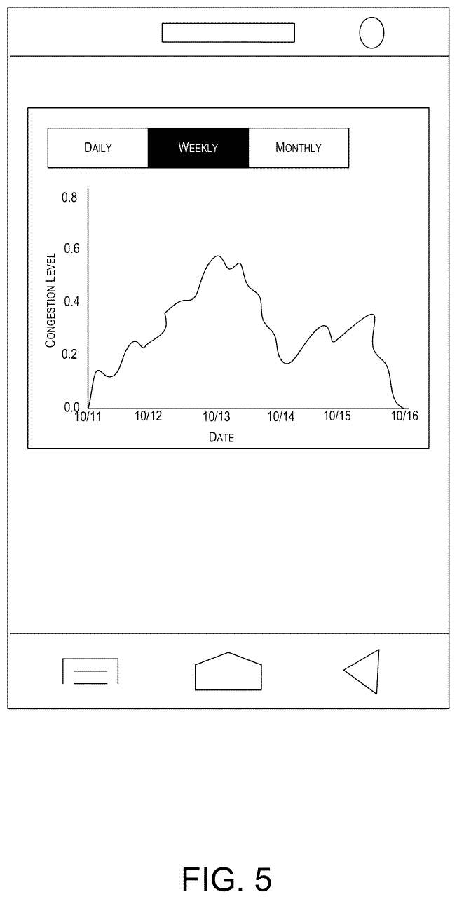

The mobile application's congestion mapping algorithm 202 interprets these fluctuations to create a real-time congestion map 204, such as a heatmap chart. Users can view the map 204 on the application's GUI 203, with the areas indicating significant blockage. As users continuously irrigates the liquid, the application 201 stores historical data for enabling long-term tracking of congestion patterns. The AI predictive analysis module continuously monitors the stored data to identify trends, provide feedback, and recommend irrigation schedules based on the user's congestion history. The mobile application 201 allows users to view the congestion history, an example of which is shown in FIG. 5. In cases of unusual or severe congestion patterns, the AI predictive analysis module alerts the user to potential health concerns.

The advantages of the present invention include providing users with visual feedback on nasal congestion in real-time, while allowing them to assess the effectiveness of each irrigation session; tracking congestion patterns over time, while aiding in chronic respiratory condition management and helping users monitor the effectiveness of their treatment; through AI-driven predictive analysis, the system provides personalized feedback and alerts, while enhancing the user's ability to proactively manage their nasal health; and enables users to take preventive action based on congestion trends, while potentially reducing the need for medical interventions in cases of chronic nasal issues.

It will finally be understood that the disclosed embodiments are presently preferred examples of how to make and use the claimed invention, and are intended to be explanatory rather than limiting the scope of the invention as defined by the claims below. Reasonable variations and modifications of the illustrated examples in the foregoing written specification and drawings are possible without departing from the scope of the invention as defined in the claim below. It should further be understood that to the extent the term “invention” is used in the written specification, it is not to be construed as a limited term as to number of claimed or disclosed inventions or the scope of any such invention, but as a term which has long been conveniently and widely used to describe new and useful improvements in technology. The scope of the invention supported by the above disclosure should accordingly be construed within the scope of what it teaches and suggests to those skilled in the art, and within the scope of any claims that the above disclosure supports. The scope of the invention is accordingly defined by the following claims.

This application is intended to cover any adaptations or variations of the present invention. Therefore, it is manifestly intended that this invention be limited only by the claims and the equivalents thereof.

1. A nasal irrigation monitoring and congestion mapping system comprising:
an electrical nasal irrigation device, the device comprising:
a water reservoir configured to store a saline solution;
a pump connected to the water reservoir to deliver the saline solution from the reservoir to the nasal passages of a user; and
an electronic pump circuit, the circuit comprising:
a microcontroller unit (MCU) configured to control the operation of the pump and pressure modes; and
a pressure detection sensor connected to the MCU to measure the flow rate of saline solution to identify pressure fluctuation data in the nasal passages during saline delivery, and provide real-time data on nasal congestion levels;
a mobile application communicatively connected to the nasal irrigation device, the mobile application comprising:
congestion mapping algorithm to interpret the pressure fluctuation data to assess a degree of nasal congestion, and map the congestion severity as nasal congestion data based on the degree of nasal congestion; and
a graphical user interface (GUI) configured to display the mapped nasal congestion data as a heatmap representing areas of high nasal blockage.

2. The system of claim 1, wherein the pressure detection sensor measure the flow rate of saline solution in the nasal passages during saline delivery being pumped through the nasal passages.

3. The system of claim 1, wherein the degree of nasal congestion includes higher or lower congestion levels.

4. The system of claim 1, wherein the algorithm assesses the degree of nasal congestion by extracting features from the pressure fluctuation data.

5. The system of claim 4, wherein the features comprising:
pressure amplitude, representing the intensity of airflow, wherein the lower amplitudes correlate with greater nasal obstruction;
pressure gradient representing the rate of pressure change over time to determine the level of resistance to airflow; and
frequency of breathing cycles to identify irregular patterns, using the timing between pressure peaks and troughs, that may indicate impaired nasal airflow.

6. The system of claim 1 is connected to a stable power supply to transmit power to the electronic pump circuit by providing consistent voltage and reducing signal noise to enable accurate congestion measurement.

7. The system of claim 1, wherein the electronic pump circuit further comprises an amplifier configured to amplify nasal congestion data signals to enhance data precision for accurate digital interpretation by the MCU.

8. The system of claim 1, wherein the GUI displays the heatmap highlighting areas of significant nasal congestion and providing visual feedback on sinus blockage severity.

9. The system of claim 1, wherein the mobile application further comprises a database configured to store historical congestion data for long-term congestion tracking.

10. The system of claim 9, wherein the mobile application further comprises an artificial intelligence (AI) predictive analysis module connected with the database to analyze congestion data trends over time, and to predict potential nasal health conditions and suggest preventive measures.

11. The system of claim 8, wherein the AI predictive analysis module generates personalized feedback, including recommended irrigation schedules, and alerts the user regarding significant congestion patterns and potential treatment needs.
